(19) United States
(12) Patent Application Publication
(10) Pub. No.: US 2021/0393855 A1
(43) Pub. Date: Dec. 23, 2021

(54) NEW HYDROGELS HAVING A SILYLATED STRUCTURE, AND METHOD FOR OBTAINING SAME

(71) Applicants: Universite de Montpellier, Montpellier (FR); Centre National de la Recherche Scientifique (CNRS), Paris (FR)

(72) Inventors: Jean Martinez, Caux (FR); Ahmad Mehdi, Montpellier (FR); Gilles Subra, Saint Gely Du Fesc (FR); Said Jebors, Jacou (FR); Cécile Echalier, Montpellier (FR)

(73) Assignees: Universite de Montpellier, Montpellier (FR); Centre National de la Recherche Scientifique (CNRS), Paris (FR)

(21) Appl. No.: 17/358,554

(22) Filed: Jun. 25, 2021

Related U.S. Application Data
(62) Division of application No. 15/743,319, filed on Jan. 10, 2018, now abandoned.

(30) Foreign Application Priority Data
Jul. 10, 2015 (FR) 1556628

(51) Int. Cl. A61L 27/52; A61L 27/54; G01N 33/543; C07F 7/08; A61L 26/00; C07F 7/18; A61L 27/18; A61L 27/22; C07K 1/107; C07K 5/072; C07K 5/097; C07K 7/06; C08G 65/336; C12N 5/0775

(52) U.S. Cl. CPC A61L 27/52; C12N 2533/50; G01N 33/5436; C07F 7/087; A61L 26/0066; A61L 26/008; C07F 7/1804; A61L 27/18; A61L 27/22; C07K 1/1077; C07K 5/06095; C07K 5/0823; C07K 7/06; C08G 65/336; C12N 5/0668; A61L 2300/404; A61L 2400/06; C12N 2533/30; A61L 27/54

(57) ABSTRACT

The present invention relates to hydrogels prepared using silylated organic molecules (such as silylated biomolecules), a process for obtaining the same, and uses thereof.

The present invention relates to hydrogels prepared using silylated organic molecules (such as silylated biomolecules), a process for obtaining same, and uses thereof.

### INTRODUCTION

There is a continuous search for new, functionalizable hydrogels having precise physicochemical or biological properties, in particular for human medicine.

Currently, however, there are many technical constraints with these materials. For example, to obtain materials having biological properties, biomolecules (peptides, proteins, saccharides, oligonucleotides, etc.) are often introduced into these materials noncovalently. This approach is indeed a definite technique for adding active molecules to hydrogels which thus exhibit, from the outset, the desired biological and rheological properties. Nevertheless, it has the disadvantage that the hydrogels thus obtained cannot be envisaged for use in vivo, for example placed in a patient, without release of the active molecule(s) incorporated within their matrix. Moreover, since the hydrogels are already formed and highly viscous (in the best case), their administration via injection is very painful for the patient.

Thus, if it is desired that the hydrogel retains biological activity without untimely release, the bond between the active agent and the hydrogel matrix must be covalent. However, to covalently bond biomolecules in these hydrogels without fundamentally altering the physicochemical properties thereof remains difficult for several reasons: since biomolecules are generally molecules the synthetic chemistry of which is often specific (e.g., molecules often having many reactive chemical functions), it is difficult not to involve secondary reactions during formation of the functionalized hydrogel. Moreover, it is difficult to covalently bond these biomolecules (which are often bulky) to the matrix core without altering the physicochemical properties and/or the integrity thereof when direct functionalization of the hydrogel is carried out.

Patent document WO2011089267 concerns, among other subjects, the production of hydrogels to which active biomolecules are covalently linked. To that end, silicon chemistry was selected in addition to the selected biomolecules. Thus, two types of silylated biomolecules are synthesized, one having the role of structural matrix of the hydrogel (in particular silylated HPMC in WO2011089267), and the other having the role of active biological functionalization of the hydrogel. The invention described in WO2011089267 thus has appeal. However, the disclosed invention does not solve all the technical problems mentioned above.

On the one hand, the rheological properties of hydrogels are difficult to control by the technique of WO2011089267. Indeed, WO2011089267 discloses hydrogels formed from a “biomolecule” having a single silylated group, the properties of which will substantially depend on the nature of the “biomolecule” fragment. This is a handicap because it is important to be able to precisely control the chemical nature of the hydrogel matrix independently of its rheological properties. It is also essential that the hydrogel ensures, for example, biocompatibility according to the model studied and/or has particular desired biological and/or physicochemical functions according to the field concerned. One aspect of the present invention was thus to facilitate the control of the rheology of hydrogels.

Moreover, only one process for producing synthetic intermediates (silylated biomolecules) is given in WO2011089267 (example 1 of said document) and involves the suspension in a solvent such as anhydrous acetonitrile of the biomolecule of interest to be silylated. This does not make it possible, therefore, to obtain all the desired silylated biomolecules. Purification thus appears to be a genuine problem which is virtually ignored in WO2011089267. Indeed, the purification described involves successive washings of the solid suspended in acetonitrile in order to remove all the impurities therefrom. However, such purification does not make it possible to obtain a product (active ingredient) of a purity necessary for medical use, and in addition requires that the product obtained is also insoluble in the washing solvent (anhydrous acetonitrile in WO2011089267). Common purification techniques such as reversed-phase chromatography (technique enabling optimal purification for most biomolecules) seem to be excluded from the technology of WO2011089267 because the silylated molecules described are highly reactive in aqueous medium, thus resulting in polymerization once introduced into an HPLC column in the presence of water.

Thus, the teaching of WO2011089267, appealing on initial examination, does not make it possible to work precisely with hydrogel components in order to provide functionalized hydrogels of medical grade or of a sufficient grade for related fields (biomedical research in particular).

However, even if there are other examples of silylated organic molecules in the literature, such as those found in WO2013190148 wherein silylated peptide conjugates are disclosed, the use thereof for producing hydrogels is not disclosed, nor is an optimal purification method involving, for example, reversed-phase HPLC. Moreover, only “xerogels”, non-hydrated forms of gels, are disclosed on that occasion. However, the control of the rheology of hydrogels is also dependent on their hydration.

In short, hydrogels have a considerable potential for use, in particular in the medical field. Nevertheless, this potential is currently limited by poor control of the production of these hydrogels, a lack of knowledge regarding the means for controlling the rheological properties of these hydrogels, or simply the impossibility of producing active biocompatible hydrogels the activity of which endures over time.

Thus, the object of the present patent application is to provide at least one technical solution to overcome the incomplete methods of the prior art in order to lead to the production of easily modifiable hydrogels, which in particular may be used in the medical and related fields. In order to overcome the rheological problems of hydrogels, while making it possible to integrate the desired (bio)molecules into the structure thereof, it was surprisingly discovered that by working with at least bi-silylated (bio)molecules, it was possible to modify the rheological properties of gels virtually independently of the nature of the so-called structural (bio)molecules. Thus, a simple model of polyethylene glycol (PEG) integrated into a hydrogel made it possible, depending on the length of the PEG, to obtain a biocompatible hydrogel with the desired rheological properties. It is indeed the size of the mesh formed by the matrix which enables the latter in the particular case of hydrogels to absorb more or less water and to give it the desired rheological properties. Moreover, by modifying the number of silylated groups introduced on the structural (bio)molecules, it is possible to precisely vary the rheological properties of the hydrogel virtually independently of the nature of these structural (bio)molecules.

Another aspect of the present invention concerns the techniques for purifying optionally functionalized hydrogel precursors. The first of these techniques involves a series of precipitations, in particular in an inert solvent such as diethyl ether. This technique indeed has the advantage that most functionalized synthetic hydrogel precursors are insoluble in ether and are not reactive in this solvent. By a succession of ether washes and optionally dissolutions in an adequate solvent, it is possible to provide functionalized synthetic hydrogel precursors of a purity sufficient for simple (inexpensive) molecules.

The present invention further provides a method of purification by reversed-phase HPLC involving a particular choice of substituents of the Si atom. Indeed, the use of a single reactive group, such as a halogen or a hydroxyl group or a hydroxyl precursor, on the Si atom allows purification on a reversed-phase HPLC column of the synthesized silylated biomolecule, whereas the techniques of the prior art remain silent on this subject. The advantage of being able to use reversed-phase HPLC is to make it possible to obtain active molecules, such as biomolecules, with an optimum degree of purity, necessary in the context of medical use.

The choice of the groups borne by the Si atom makes it possible to avoid in most cases unwanted secondary reactions. The potential of the present invention thus also lies in the fact that it would seem that any biomolecule of biological interest (in particular a peptide sequence) can be introduced in a controlled manner into the hydrogels according to the present invention in order to endow them with biological properties.

Moreover, it was found in a completely opportune manner that the novel physicochemical properties of a bi-silylated polyethylene glycol hydrogel allow very easy grafting of silylated biomolecules. The bi-silylated polyethylene glycol hydrogel thus formed is biocompatible, biodegradable, completely synthetic, and easily modifiable in terms of hydration (more or less easily impregnated by liquids, for example aqueous liquids). Moreover, it was discovered that the (bi-silylated) PEG hydrogel matrix can be formed with or without the presence of water, which allows great versatility in terms of the use of this polymer (dehydrated hydrogels which can be subsequently hydrated). Nevertheless, in order to obtain a structurally homogeneous hydrogel (homogeneity of structure and hydration), it is preferable to form the hydrogel in a mostly aqueous medium. Moreover, the possibility of modifying the reactivity of the silyl group by decreasing or increasing the number of reactive substituents thereupon (1, 2 or 3 reactive substituents), makes it possible to precisely control the degree of cross-linking of the hydrogel and thus to be able to precisely control the rheology thereof.

Another aspect of the present invention concerns the insertion of liquid hydrogels into the organism with in situ polymerization of these hydrogels. Indeed, a recurring problem of liquid hydrogels prepared in advance is that they are difficult to inject because of their viscosity, which, during local administration, causes the patient a varying degree of acute pain. The technique of WO2011089267, even if it is mentioned in this document, cannot be used as such because the purity of the silylated biomolecules is inadequate for a medical application with injection in liquid form and in situ gelling. The purification methods mentioned above make it possible to overcome this technical problem.

Indeed, hydrogels can be prepared by simple dissolution of hybrid blocks in phosphate buffer then incubation of the solution at 37° C., which makes it possible to envisage the in vivo injection thereof with in situ gelling.

### SUMMARY OF THE INVENTION

The object of the present invention concerns a process for producing a hydrogel comprising the steps of:
a) sol-gel polymerization of at least one molecule of formula (I):

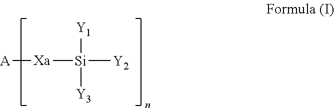

wherein:
   n is an integer greater than or equal to 2; preferentially less than 10, more preferentially less than 5 or 4.
   A is a structural organic polymer, preferentially of synthetic origin (which may be, for example, selected from proteins, peptides such as collagen derivatives, in particular the sequences comprising Pro-Hyp-Gly or Pro-Pro-Gly or Asp-Pro-Gly or Pro-Lys-Gly tripeptide repeats, self-assembly peptide sequences such as Arg-Ala-Asp-Ala, oligoprolines, oligoalanines, polysaccharides, such as hyaluronic acid and derivatives thereof, oligonucleotides, C₁-C₆-alkylene-glycol polymers, or polyvinylpyrrolidone);
   Xa is a chemical bond or a spacer group preferentially represented by a divalent radical derived from a saturated or unsaturated aliphatic hydrocarbon chain comprising from 1 to 10 carbon atoms, optionally intercalated with one or more structural linkers selected from arylene or fragments —O—, —S—, —C(═O)—, —SO₂— or —N(R₁)—, wherein said chain is unsubstituted or is substituted by one or more radicals selected from halogen atoms, a hydroxyl group, a C₁-C₄ alkyl group, a benzyl group and/or a phenethyl group;
   R₁ represents a hydrogen atom, an aliphatic hydrocarbon group comprising from 1 to 6 carbon atoms, a benzyl or a phenethyl;
   Y₁, Y₂, Y₃, which may be identical or different, each independently represents a hydrogen atom, a halogen atom, an —OR₂ group, an aryl or a saturated or unsaturated aliphatic hydrocarbon chain comprising from 1 to 6 carbon atoms optionally substituted by a halogen atom, an aryl group or a hydroxyl group;
   R₂ represents a hydrogen atom, an aryl group or a saturated or unsaturated aliphatic hydrocarbon chain comprising from 1 to 6 carbon atoms;
   wherein at least two Xa groups as defined above are linked to different attachment points on A;
b) mixing with water, optionally at the same time as step a); preferentially wherein the water is medical grade, and
c) recovering the hydrogel.

The process for producing a hydrogel as defined above may further comprise the addition, at the same time as or subsequent to step a), of at least one type of molecule of formula (II):

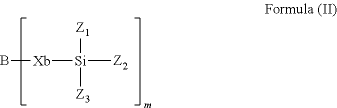

wherein:
   m is an integer greater than or equal to 1; preferentially m is less than 10, more preferentially less than 5 or 4, even more preferentially equal to 1;
   B is an active ingredient, preferentially a biomolecule or a fluorophore (which may be, for example, selected from a peptide, an oligopeptide, a protein, such as collagen, a deoxyribonucleic acid, a ribonucleic acid, a polysaccharide, such as a pectin, a chitosan, a hyaluronic acid, a polyarabinose and polygalactose polysaccharide, and a glycolipid);
   Xb is a chemical bond or a spacer group preferentially represented by a divalent radical derived from a saturated or unsaturated aliphatic hydrocarbon chain comprising from 1 to 10 carbon atoms, optionally intercalated with one or more structural links selected from arylene or fragments —O—, —S—, —C(═O)—, —SO₂— or —N(R₃)—, wherein said chain is unsubstituted or is substituted by one or more radicals selected from halogen atoms, a hydroxyl group, a C₁-C₄ alkyl group, a benzyl group and/or a phenethyl group;
   R₃ represents a hydrogen atom, an aliphatic hydrocarbon group comprising from 1 to 6 carbon atoms, a benzyl or a phenethyl;
   Z₁, Z₂, Z₃, which may be identical or different, each independently represents a hydrogen atom, a halogen atom, an —OR₄ group, an aryl or a saturated or unsaturated aliphatic hydrocarbon chain comprising from 1 to 6 carbon atoms optionally substituted by a halogen atom, an aryl group or a hydroxyl group;
   R₄ represents a hydrogen atom, an aryl group or a saturated or unsaturated aliphatic hydrocarbon chain comprising from 1 to 6 carbon atoms; and
   wherein preferentially only one of the Z₁, Z₂, or Z₃ groups is a halogen atom or an OR₄ group.

The object of the present invention thus concerns a hydrogel that can be obtained by the process as defined herein.

The object of the present invention further relates to a hydrogel according to the present invention for therapeutic and/or surgical use, preferentially characterized in that said hydrogel allows the delivery and/or the transport of active molecules, or for use in vivo in tissue engineering which may, for example, be achieved by in situ polymerization of said hydrogel in a living organism following the casting or the injection of molecules of formula (I) and optionally (II) as defined above.

Thus, the present invention concerns a hydrogel that can be obtained by the process according to the present invention for use as defined above, characterized in that the polymerization of said hydrogel is carried out in situ in a living organism, i.e., an animal such as a mammal like man, following the casting or the injection, preferentially painless or relatively painless, of molecules of formula (I) and optionally (II) as defined above.

Another object of the present invention concerns the in vitro use of a hydrogel according to the present invention in tissue engineering.

Moreover, the present invention concerns a process for purifying a product of formula (I) or (II) as defined herein, characterized in that said process comprises the following steps:
a1) optionally solubilizing in a solvent the product of formula (I) or (II) to be purified;
b1) precipitating the product of formula (I) or (II) to be purified, optionally in solution according to step a1), in a suspension liquid, i.e., in which the products (I) or (II) are not soluble;
c1) filtering the solid obtained in step (b1);
d1) repeating at least once steps a1) if necessary, b1) and c1); and
e1) recovering the purified product of formula (I) or (II).

The object of the present invention further relates to a process for purifying a product of formula (II) as defined herein wherein only one of the Z₁, Z₂, Z₃ groups is a halogen atom or an —OR₃ group, characterized in that said process comprises the following steps:
a2) passing the product of formula (II) to be purified through a liquid-phase chromatography column, i.e., in an elution solvent, preferentially in reversed-phase;
b2) evaporating the elution solvent of step a2), optionally by freeze-drying; and
c2) recovering the purified product of formula (II).

Thus, the present invention also concerns a product of formula (I) or (II) as defined herein, which can be obtained by the purification process of the invention, characterized in that said product has a purity of at least 98% by mass relative to the total weight of product, preferentially greater than 99% by mass.

### Definitions

“Hydrogel”

A “hydrogel” is a type of material comprising an aqueous liquid component and a solid component. Structurally, it is composed of a matrix of polymer chains, swollen by a fluid comprising water, this fluid, preferentially consisting primarily of water, being preferentially in a proportion greater than or equal to 40% of said hydrogel by weight, more preferentially ranging between 40% and 99% by weight, 50% and 98%, 60% and 97%, 70% and 96% or between 80% and 95%. Preferably, this fluid comprises at least 95% water by weight, even 100% water. This fluid may in addition be a liquid of biological origin. The water content of a hydrogel also chiefly determines the hydrogel's physicochemical characteristics. These hydrogels can also be found in various biomedical applications, notably in the release of medicinal products and the treatment of skin burns.

“Polymer that can be Cross-Linked” or “Cross-Linkable Polymer”

The hydrogels according to the invention consist of polymer chains linked by covalent bonds. A cross-linked polymer comprises nodes with at least 3 preferentially covalent chemical bonds. This cross-linking allows the hydrogels according to the present invention to provide a so-called “permanent” character to the cross-linking nodes because the tetravalent silicon atom generates 4 covalent bonds. Nevertheless, owing to the dual nature (organic and inorganic) of the hydrogels according to the present invention, it is possible to obtain remarkable properties: partial stiffness and a capacity to give an elastic response to mechanical stress. The organic nature allows the hydrogels produced to be highly biocompatible and non-toxic. Moreover, the hydrogels according to the present invention have a degree of flexibility very similar to that of natural tissues (induced by their high water content). The term “cross-linkable” thus preferentially refers to the capacity of the silicon atoms to generate at most 3 covalent bonds with at most 3 groups comprising an Si atom (such as Si—OH).

“Sol-Gel Polymerization”

The sol-gel process makes it possible to produce an inorganic polymer by simple chemical reactions at a temperature close to room temperature (in actual fact applicable to temperatures ranges of 0° C. to 150° C., preferentially between 20° C. and 70° C., more preferentially between 35° C. and 40° C.). Typically, the synthesis is carried out starting with alkoxysilanes or silanols of formula Si(OR)ₙ where R is a CₙH₂ₙ₊₁ alkyl organic group or a hydrogen.

One of the advantages of this process is that these precursors are either liquid or solid; in this case they are, for the most part, soluble in common solvents. It is thus possible to prepare homogeneous mixtures of monomers (precursors) or oligomers.

The simple chemical reactions on which the process is based are initiated when the precursors are mixed with water: hydrolysis of the typically alkoxy groups (or halogen as the case may be) occurs first, then condensation of the hydrolyzed products leads to gelation of the system, thus forming the hydrogel.

“Structural Organic Polymer”

According to the present invention, the term “structural organic polymer” refers to a polymer of organic nature, therefore a hydrocarbon polymer, which makes it possible to structure the hydrogel in the form of a polymer matrix. This is easily achieved as soon as the monomers making up said structural organic polymer are controlled in terms of chemical structure and of purity; i.e., by common chemical synthesis or purification techniques. Indeed, by controlling the nature of the monomers involved in the polymerization of the hydrogel, it is possible to vary the physicochemical and rheological properties of the polymer ultimately obtained.

“Synthetic Origin”

The raw materials, such as petroleum, used for the production of synthetic materials are obviously derived from nature. However, the synthetic materials created by humans by means of chemical processes, differentiate them from other materials. According to the present invention, the expression “synthetic origin” implies that the product concerned has been modified at least once by a chemical process developed by humans.

“Chemical Bond”

Any attractive interaction which maintains at least two atoms a short distance apart is called a “chemical bond”. This interaction may be directional, such as the bond between two atoms within a molecule, or non-directional, such as the electrostatic interaction which maintains in contact the ions of an ionic crystal. It may be strong as in the two preceding examples, or weak as in the van der Waals interactions which are of dipolar nature.

“Spacer Group”

According to the present invention, a fragment comprising at least one atom is called a “spacer group”. Preferentially, the spacer group contains at least one carbon atom. Advantageously, the spacer group makes it possible to move two chemical groups apart within the same molecule, to decrease steric hindrance between group A (or B) and the Si atom. More advantageously, the spacer group allows the silylated group to react with limited hindrance of fragment A (or B) or allows group A (or B) to interact and to retain the biological properties thereof, with limited hindrance of the Si-containing fragment. Moreover, the spacer group allows a stable bond between fragment A (or B) and Si, while allowing the silicate fragment to react. It is thus clear that the spacer group cannot be regarded as a constituent part of fragment A (or B), such as for example an amino acid residue if A (or B) is a peptide fragment.

Advantageously, the spacer group comprises, or consists of, a saturated or unsaturated aliphatic hydrocarbon chain, preferentially comprising between 1 and 10 carbon atoms, more preferentially between 2 and 5 carbon atoms. Preferably, the spacer group is a saturated aliphatic hydrocarbon chain.

The spacer group (in particular when it is a saturated or unsaturated aliphatic hydrocarbon chain as described above) may further include heteroatoms, in particular selected from N, O, S, or P, and in addition may be substituted, in particular by halogen atoms, or by hydroxyl, aryl, C₁-C₄ alkyl, sulfate, amine or phosphate groups. However, if heteroatoms are present in the spacer group, preferably these heteroatoms are not directly linked to Si.

Preferably, the spacer group is a linear or branched C₁-C₄ alkyl fragment. More preferably, the spacer group comprises a —(CH₂)₂—, —(CH₂)₃—, —(CH₂)₄— fragment. Even more preferably, the spacer group comprises the fragment —(CH₂)₃—.

“Derived Divalent Radical”

According to the present invention, the expression “derived divalent radical” concerns an element having a valence of two. Valence is the number of chemical bonds formed, which may be covalent, polar or ionic bonds. The term “derived” simply refers to the chemical bonds formed to incorporate said radical into the structure.

“Saturated or Unsaturated Aliphatic Hydrocarbon Chain”

The expression “saturated or unsaturated aliphatic hydrocarbon chain” refers to fragments of type C₁-C₁₀ alkyl, C₂-C₁₀ alkene or C₂-C₁₀ alkyne; preferentially these chains are linear or branched.

“C₁-C₁₀ Alkyl”

In the present invention, the term “C₁-C₁₀ alkyl” or “alkyl of 1 to 10 carbon atoms” refers to a linear, branched or cyclic saturated aliphatic group comprising from 1 to 10 carbon atoms, such as for example a methyl, ethyl, isopropyl, tert-butyl, n-pentyl, cyclopropyl, cyclohexyl group, etc.

“C₂-C₁₀ Alkene”

In the context of the present invention, the term “C₂-C₁₀ alkene” group or “alkene of 2 to 10 carbon atoms” refers to a linear, branched or cyclic mono- or poly-unsaturated aliphatic group comprising from 2 to 10 carbon atoms. An alkene group according to the invention preferably comprises one or more ethylenic unsaturations. By way of example, mention may be made of ethylene, propylene, propyl-2-ene or propyl-3-ene, butylene, cyclobutene groups, etc.

“C₂-C₁₀ Alkyne”

In the context of the present invention, the term “C₂-C₁₀ alkyne” group or “alkyne from 2 to 10 carbon atoms” refers to a linear, branched or cyclic aliphatic group comprising from 2 to 10 carbon atoms and at least one double unsaturation, i.e., a triple bond between two carbon atoms. An alkyne group according to the invention preferably comprises one or more double unsaturations. By way of example, mention may be made of acetylene, propyne, butyne groups, etc.

“Aryl”

The term “aryl” group refers to an aromatic group preferably comprising from 5 to 10 carbon atoms, comprising one or more rings and optionally comprising one or more heteroatoms, in particular oxygen, nitrogen or sulfur, such as, for example, a phenyl, furan, indol, pyridine, naphthalene group, etc.

“Arylene”

An “arylene” group represents a substituent of an organic compound derived from an aryl fragment wherein at least one hydrogen atom has been removed from two carbons included in the aryl. Preferentially, it is a phenethyl group.

“Phenethyl”

The term “phenethyl” represents the fragment:

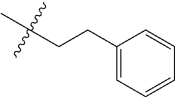 [SMILES: *CCc1ccccc1]

“Halogen”

The term “halogen” refers to the chemical elements of the 17^{th} column of the periodic table, formerly called group VII or VIIA. These chemical elements are preferentially: fluorine, chlorine, bromine and iodine.

“Hydroxyl”

According to the present invention, the term “hydroxyl” refers to the fragment —OH, and optionally the salts thereof, for example the sodium or potassium salts.

“Xa Linked to Different Attachment Points on A”;

According to the present invention, the expression “Xa linked to different attachment points on A” means that the Xa groups are not linked to the same atom belonging to polymer A. Preferably, the attachment points (i.e., the attachment atoms) are positioned as distant apart as possible on polymer A. One way to evaluate this distance may be simply to count the number of atoms between the two attachment points on polymer A. Indeed, the physicochemical and rheological properties are more easily controllable when the entropy of polymers A is decreased, which is achieved in theory by fixing the ends of the polymer chains.

“Recovery”

According to the present invention, the term “recovery” means that the products obtained are extracted according to the common techniques of the art, i.e., by means of biphasic washing comprising for example an organic solvent and water; alternatively, it is possible to recover the products by suspension in the liquid which contains them, then filtering them. Another way of recovering the products may be quite simply to evaporate or freeze-dry the solvent which contains them. This recovery phase may further include purification by washing or passage through a chromatographic column, if need be.

“Medical Grade Water”

According to the present invention, the term “medical grade water” means that the water is of ultrapure grade commonly used in the medical field, optionally with adjuvants for matching the physiological conditions of the human body, such as salts like NaCl, KCl, CaCl₂, MgCl₂, etc.; pH buffer such as pH 7.4 phosphate buffers, etc.; or sugars such as glucose, mannitol.

“Active Ingredient”

The general definition of an active ingredient is: the active ingredient is the molecule which, in a medicinal product, has a therapeutic effect. In the context of the present invention, which concerns hydrogels, the active ingredient is the molecule having the distinctive physicochemical or biological properties of the polymer which carries it. For example, the active ingredient may be a known medicinal product, a biological molecule such as a peptide sequence allowing the attachment of biological cells, or a dye or any other molecule having one or more biological or physicochemical activities. The active ingredient may be of natural or synthetic origin.

Advantageously, the hydrogel according to the present invention comprises an active ingredient with antimicrobial, antibiotic and/or antifungal activity.

Preferentially, the active ingredient is a biomolecule, for example selected from a peptide, a protein, a glycopeptide, a glycoprotein, a deoxyribonucleic acid, a ribonucleic acid, a pectin, a chitosan, a hyaluronic acid, a saccharide, an oligosaccharide, a lipid, a glycolipid, or derivatives thereof.

Advantageously, the active ingredient is selected from molecules that promote cell adhesion, such as peptides containing or consisting of sequence ArgGlyAsp, in particular, peptide H-GlyArgGlyAspSerPro-OH (Seq ID 1), molecules that promote healing, such as peptides containing or consisting of sequence H-GluGlyLeuGluProGly-OH (Seq ID 2), molecules that promote the production of extracellular matrix such as peptides containing or consisting of H-ValGlyValAlaProGly-OH (Seq ID 3) or antibacterial molecules, such as peptides containing or consisting of H-AhxArgArg-NH₂, pain-killing molecules (e.g., peptides), molecules (e.g., peptides) that promote blood coagulation, anticoagulant molecules (e.g., peptides), antiproliferative molecules (e.g., peptides), or a mixture of several of these (bio)molecules.

Advantageously, the active ingredient may be a dye, a fluorophore or a marker selected from the following compounds: fluorescein, fluorescein sodium salt, 4′,5′-Bis[N,N-bis(carboxymethyl)-aminomethyl]fluorescein, 6-[fluorescein-5(6)-carboxamido]hexanoic acid, 6-[fluorescein-5(6)-carboxamido] hexanoic acid, fluorescein-5(6)-isothiocyanate N-hydroxysuccinimide ester, fluorescein-α-D-N-acetylneuraminide-polyacryl-amide, fluorescein amidite, fluorescein-di(β-D-galactopyranoside), fluorescein-di-(β-D-glucopyranoside), fluorescein diacetate, fluorescein-5(6)-isothiocyanate diacetate, fluorescein-5-maleimide diacetate, fluorescein-6-isothiocyanate diacetate, fluorescein dibutyrate, fluorescein dilaurate, fluorescein diphosphate triammonium salt, fluorescein-hyaluronic acid, fluorescein isothiocyanate-Dextran 500000-Conjugate, fluorescein isothiocyanate isomer I, fluorescein-dextran isothiocyanate, mercury-fluorescein acetate, mono-p-guanidinobenzoate-fluorescein hydrochloride, —O,O′-fluorescein diacrylate, fluorescein O,O′-dimethacrylate, fluorescein O-acrylate, fluorescein O-methacrylate, fluorescein N-hydroxysuccinimide ester, fluorescein-5-thiosemicarbazide, fluorescein-α-D-galactosamine polyacrylamide, fluorescein-α-D-mannopyranoside-polyacrylamide, 4(5)-(iodoacetamido)-fluorescein, 5-(Bromomethyl)fluorescein, 5-(Iodoacetamido)fluorescein, 5-Carboxy-fluorescein diacetate N-succinimidyl ester, 6-Carboxy fluorescein diacetate N-succinimidyl ester, Aminophenyl-fluorescein, Biotin-4-fluorescein, hydroxyphenyl-fluorescein, MTS-4-fluorescein, poly(fluorescein-isothiocyanate allylamine) hydrochloride, poly(fluorescein-O-acrylate), poly(fluorescein-O-methacrylate), PPHT-fluorescein acetate, 5-([4,6-dichlorotriazin-2-yl]amino) fluorescein hydrochloride, 6-([4,6-dichlorotriazin-2-yl]amino) fluorescein hydrochloride, poly[(methylmethacrylate)-co-(fluorescein-O-methacrylate)], poly[methylmethacrylate-co-(fluorescein-O-acrylate)], 5(6)-(Biotinamidohexanoylamido)pentylthioureidylfluorescein, N-(5-fluoresceinyl)maleimide, Mercury-dibromo-fluorescein disodium salt, fluorescein-di-[methylene-N-methylglycine], 2′,4′,5′,7′-tetrakis-(acetoxymercuro)-fluorescein disodium salt, erythrosine B, ethyl eosin, 5-carboxy fluorescein, 5-carboxy fluorescein N-succinimidyl ester, octadecyl rhodamine B, 6-Carboxy-fluorescein N-hydroxysuccinimide ester, dibenzyl fluorescein, rhodol, 6-amino fluorescein, rhodamine 6G, rhodamine B or rhodamine 123. These dyes, fluorophores and/or markers can be incorporated into the hydrogel with a biomolecule or a mixture of several biomolecules.

The term “biomolecule” according to the present invention relates to a molecule which can be found in the biological environment, i.e., synthesized by a living organism. This definition also includes the functional analogues of these molecules (which thus become synthetic molecules). Indeed, it is for example common in the art to modify the structure of biomolecules in order to keep only the active region or the active site thereof, or to add protective groups for the reactive functions, thus preventing secondary reactions, for example.

These protective groups and the use thereof are described in works such as, for example, Greene, “Protective Groups in Organic Synthesis”, Wiley, New York, 2007 4^{th} edition; Harrison et al. “Compendium of Synthetic Organic Methods”, Vol. 1 to 8 (J. Wiley & Sons, 1971 to 1996).

“Antimicrobial Activity”

“Antimicrobial activity” according to the present invention is the generic definition as understood by the person skilled in the art, i.e., an effect relating to an antimicrobial agent. An antimicrobial (agent) is a substance that kills, slows the growth of or blocks the growth of one or more microbes. In the context of the present invention, the term “growth” refers to any cellular operation allowing the cell to increase in volume, allowing the cell to divide or allowing the cell to reproduce. A microbe in the context of the present invention is any unicellular or multicellular organism pathogenic or parasitic to other living organisms such as humans.

For example, the antimicrobials may be generally selected from the various following families: beta-lactams, cephalosporins, fosfomycin, glycopeptides, polymyxins, gramicidins, tyrocidine, aminosides, macrolides, lincosamides, synergistins, phenicols, tetracyclines, fusidic acid, oxazolidinones, rifamycins, quinolones, fluoroquinolones, nitrated products, sulfamides, trimethoprim, and mixtures thereof. More specifically, the antimicrobials may be selected from nystatin, miconazole nitrate, lauryl-oxypropyl-β-aminobutyric acid, amphotericin B, undecylenic acid, chlorquinaldol, econazole nitrate, natamycin, cloprothiazole, clotrimazole, tolnaftate, lucensomycin, tetracycline, erythromycin, penicillins, oxacillin, cloxacillin, ampicillin, amoxicillin, bacampicillin, metampicillin, pivampicillin, azlocillin, mezlocillin, piperacillin, ticarcillin, pivmecillinam, sulbactam, tazobactam, imipenem, cephalexin, cefadroxil, cefaclor, cefatrizine, cefalotine, cefapirin, cefazolin, cefoxitin, cefamandole, cefotetan, cefuroxime, cefotaxime, cefsulodin, cefoperazone, cefotiam, ceftazidime, ceftriaxone, cefixime, cefpodoxime, cefepime, latamoxef, aztreonam, vancomycin, vancocin, teicoplanin, polymyxin B, colistin, bacitracin, tyrothricin, streptomycin, kanamycin, tobramycin, amikacin, sisomicin, dibekacin, netilmicin, spectinomycin, spiramycin, erythromycin, josamycin, roxithromycin, clarithromycin, azithromycin, lincomycin, clindamycin, virginiamycin, pristinamycin, dalfopristin-quinupristin, chloramphenicol, thiamphenicol, tetracycline, doxycycline, minocycline, fusidic acid, linezolid, rifamycin, rifampicin, nalidixic acid, oxolinic acid, pipemidic acid, flumequine, pefloxacin, norfloxacin, ofloxacin, ciprofloxacin, enoxacin, sparfloxacin, levofloxacin, moxifloxacin, nitroxoline, tilbroquinol, nitrofurantoin, nifuroxazide, metronidazole, ornidazole, sulfadiazine, sulfamethizole, trimethoprim, isoniazid and derivatives and mixtures thereof.

Preferentially, the grafted antimicrobials have a contact mode of action.

Moreover, all these antimicrobial molecules have variably reactive chemical functions. Thus, the person skilled in the art is quite capable of adapting the object of the present invention in order to use one of these functions as an attachment point to fragment Xb.

“Antibiotic Activity”

“Antibiotic activity” (equivalent to the term “antibacterial”) according to the present invention is the generic definition as understood by the person skilled in the art, i.e., an effect relating to an antibiotic agent. An antibiotic (agent) is a substance that kills, slows the growth of or blocks the growth of one or more bacteria. By “growth” is meant in the context of the present invention any cellular operation allowing the cell (bacterium) to increase in volume, allowing the cell (bacterium) to divide or allowing the cell (bacterium) to reproduce.

For example, the antibiotics may be generally selected from the various following families: beta-lactams, monobactams, penicillins, beta-lactamase inhibitors, aminoglycosides, glycylcycline, tetracyclines, quinolones, glycopeptides, lipopeptides, macrolides, ketolides, lincosamides, streptogramins, oxazolidinones, polymyxins.

More specifically, the antibiotics may be selected from amikacin, gentamycin, tobramycin, imipenem, meropenem, ertapenem, the compound known as PZ-601, cefazoline, cefepime, cefotaxime, cefoxitin, ceftaroline, ceftazidime, ceftobiprole, ceftriaxone, cefuroxime, cephalexin, aztreonam, amoxicillin, clavulanate, ampicillin, sulbactam, oxacillin, piperacillin, tazobactam, ticarcillin, penicillin, doxycycline, minocycline, tetracycline, tigecycline, ciprofloxacin, gatifloxacin, grepafloxacin, levofloxacin, moxifloxacin, ofloxacin, azithromycin, clarithromycin, roxithromycin, telithromycin, colistin, polymyxin B, fosfomycin, trimethoprim and sulfamethoxazole.

As examples of antibiotic agents, further mention may be made of alcohols, C₂-C₈ polyols, acetate, aluminum benzoate and diacetate, preservatives such as benzalkonium chloride, cetrimonium chloride, chlorhexidine, climbazole, citrates, silver oxide and sulfate, acids such as boric acid, usnic acid, pyroglutamic acid and derivatives, zinc acetate, borate, salicylate and sulfate, antimicrobial peptides such as beta-defensins, and mixtures thereof.

Preferentially, the grafted antibiotics have a contact mode of action.

All these antibiotic molecules have variably reactive chemical functions. Thus, the person skilled in the art is quite capable of adapting the object of the present invention in order to use one of these functions as an attachment point to fragment Xb.

“Antifungal Activity”

“Antifungal activity” according to the present invention is the generic definition as understood by the person skilled in the art, i.e., an effect relating to an antifungal agent. An antifungal (agent) is a substance that kills, slows the growth of or blocks the growth of at least one fungus. By “growth” is meant in the context of the present invention any cellular operation allowing the cell (fungus) to increase in volume, allowing the cell (fungus) to divide or allowing the cell (fungus) to reproduce.

Examples of antifungals may in addition be selected from the various following families: polyenes, imidazoles, triazoles, nucleoside analogues, allylamines, echinocandins, sordarins, morpholines, griseofulvin, ciclopirox olamine, selenium sulfide, and mixtures thereof.

More preferably, the antifungal agent is selected from nystatin, amphotericin B, ketoconazole, econazole, miconazole, clotrimazole, fluconazole, itraconazole, voriconazole, posaconazole, 5-fluorocytosine, naftifine, terbinafine, caspofungin, amorolfine, and derivatives and mixtures thereof.

Preferentially, the grafted antifungals have a contact mode of action.

All these antifungal molecules have variably reactive chemical functions. Thus, the person skilled in the art is quite capable of adapting the object of the present invention in order to use one of these functions as an attachment point to fragment Xb.

“Natural Active Ingredient”

A natural active ingredient, such as a natural biomolecule, is an active ingredient found in the environment without direct human intervention (except its extraction/isolation).

“Synthetic Active Ingredient”

A synthetic active ingredient is an active ingredient that is not found in the environment without direct human intervention (except its extraction/isolation). For example, an active ingredient such as a synthetic peptide can be a sequence of a natural peptide wherein at least one natural amino acid has been replaced by another, natural or synthetic.

“Peptide” and “Protein”

The terms “peptide” (equivalent to the term “oligopeptide”) and “protein” should be understood to mean polymers of amino acids, wherein said amino acids are linked by a peptide and/or pseudopeptide bond. A peptide generally contains between 2 and 80 to 100 amino acids, the upper limit not being clearly defined. Beyond this upper limit, one speaks rather of proteins. Preferentially, the peptide active ingredient according to the present invention contains between 2 and 80 amino acids, more preferably between 3 and 40, and even more preferably between 4 and 20.

Peptides or proteins can be extracted from a biological environment or produced synthetically. Peptide synthesis techniques are described in Paul Lloyd-Williams, Fernando Albericio, Ernest Giralt, “Chemical Approaches to the Synthesis of Peptides and Proteins”, CRC Press, 1997 or Houben-Weyl, “Methods of Organic Chemistry, Synthesis of Peptides and Peptidomimetics”, Vol E 22a, Vol E 22b, Vol E 22c, Vol E 22d., M. Goodmann Ed., Georg Thieme Verlag, 2002.

“Amino Acid”

The expression “amino acid” should be understood to mean any molecule having at least one carboxylic acid, at least one amine and at least one carbon linking said amine and said carboxylic acid. Preferentially, the amino acids which may be used in the context of the present invention are so-called “natural” amino acids and/or synthetic amino acids as defined below. Preferentially, the amino acids of the present invention are of the L-configuration.

“Natural Amino Acid”

The expression “natural amino acid” represents, among others, the following amino acids: glycine (Gly), alanine (Ala), valine (Val), leucine (Leu), isoleucine (Ile), serine (Ser), threonine (Thr), phenylalanine (Phe), tyrosine (Tyr), tryptophan (Trp), cysteine (Cys), methionine (Met), proline (Pro), aspartic acid (Asp), asparagine (Asn), glutamine (Gln), glutamic acid (Glu), histidine (His), arginine (Arg) and lysine (Lys). The preferred natural amino acids according to the present invention are the L-series amino acids.

“Synthetic Amino Acid”

The term “synthetic amino acid” refers to all the non-“coded” and non-natural amino acids as defined above.

“Carbohydrate”

The term “carbohydrate” comprises monosaccharides and polysaccharides.

A monosaccharide, or simple sugar, is a hydrated carbon polymer, the carbons of which are linked by a C—C bond. There are two types of simple sugars: aldoses and ketoses. Monosaccharides are in addition able to be “cyclized” via a hemiacetal function. The preferred monosaccharides according to the present invention are the D-series monosaccharides. Monosaccharides are classified by number of carbons. For example, the 6-carbon monosaccharides are hexoses of formula C₆H₁₂O₆ and may be allose, altrose, glucose, mannose, gulose, idose, galactose or talose. The 5-carbon monosaccharides are pentoses of formula C₅H₁₀O₅ and may be ribose, arabinose, xylose, lyxose.

A polysaccharide is a polymer made up of monosaccharides (preferentially of the D-series) joined by glycosidic bonds. Examples of polysaccharides are cellulose and derivatives thereof, pectin, chitosan, or hyaluronic acid. Cellulose derivatives include hydroxypropylmethylcellulose (HPMC), hydroxyethylcellulose (HEC), hydroxypropylcellulose (HPC), carboxymethylcellulose (CMC).

Natural or synthetic carbohydrates are included in this definition.

“Glycopeptide”

A glycopeptide is a peptide linked to a carbohydrate via at least one chemical bond.

“Glycoprotein”

A glycoprotein is a protein linked to a carbohydrate via at least one chemical bond.

“Deoxyribonucleic Acid”

Deoxyribonucleic acid, or DNA, is a biological macromolecule present in all cells and in numerous viruses. DNA contains all the genetic information, called the genotype, allowing the development and the functioning of living beings.

“Ribonucleic Acid”

Ribonucleic acid (RNA) is a biological molecule present in practically all living beings, and also in certain viruses. RNA is a molecule which is chemically very similar to DNA and, furthermore, it is generally synthesized in cells from a DNA template of which it is a copy. Living cells use RNA in particular as an intermediate for genes in order to synthesize the proteins they need.

“Pectin”

Pectins are polysaccharides characterized by an α-D-galacturonic acid backbone and small amounts of variably branched α-L-rhamnose.

“Chitosan”

Chitosan or chitosane is a polysaccharide composed of the random distribution of β-(1-4)-linked D-glucosamine (deacetylated unit) and of N-acetyl-D-glucosamine (acetylated unit).

“Hyaluronic Acid”

Hyaluronic acids are polymers of disaccharides, themselves composed of D-glucuronic acid and D-N-acetylglucosamine, linked by alternating β-1,4 and β-1,3 glycosidic bonds. Polymers of this repeating unit may have a mean size between 400 and 10⁷ Da in vivo. In the context of the present invention, polymers of this repeating unit may have a mean size between 1000 and 7·10⁶ Da, preferentially between 1500 and 5·10⁶ Da, for example between 2000 and 9000 Da or between 10⁴ and 4·10⁶, such as between 150000 and 500000 or between 2·10⁶ and 3·10⁶. For example, in the context of the present invention, the polymers of this repeating unit may have a mean size of roughly 6400 Da, 2·10⁵ Da or 2.6·10⁶ Da.

“Lipid”

Lipids include several classes of molecules comprising fatty acids, glycerides, phosphoglycerides, sphingolipids, sterols, prenols.

Fatty acids are carboxylic acids with an aliphatic chain. Glycerides consist of a glycerol residue esterified by one, two or three fatty acids, which are called monoglycerides, diglycerides and triglycerides, respectively. Phosphoglycerides are phospholipids made up of two fatty acid residues esterifying a glycerol residue, which is itself esterified by a phosphate residue. Sphingolipids consist of an aliphatic amino alcohol, produced de novo from serine and a long-chain acyl-coenzyme A, and converted into, among other things, ceramides, phosphosphingolipids and glycosphingolipids.

“Glycolipid”

A glycolipid is a saccharide which is linked, preferentially by a phosphate group, to a lipid.

“In Situ Polymerization”

According to the present invention, the expression “in situ polymerization” means that polymerization occurs after injection into a living organism, such as a human being. This is achievable when the polymerization conditions are those of the physiological environment of the living organism in question (pH, temperature, water, buffer, etc.). Introduction of the non-polymerized precursor into the living organism may be carried out simply by casting said precursor onto or into the living organism, if the latter has been first opened using surgical instruments. Alternatively, the hydrogel precursor may be introduced into the living organism by injecting it using a suitable syringe and hollow needle. The hydrogel precursor then polymerizes once in contact with said living organism. Advantageously according to the present invention, the injection is relatively painless, because the precursor may be selected to be very fluid, and thus the injection means (syringe and especially the needle diameter) may be of small size, thus promoting the introduction thereof into the living organism in a relatively painless manner. Moreover, since the liquid to be injected is less viscous than if the hydrogel had been formed beforehand, the liquid once injected takes its position much more easily, without distorting nearby tissues, resulting in a less painful injection.

### DETAILED DESCRIPTION

The exceptional physicochemical properties of the hydrogels according to the present invention enable pharmaceutical and biomedical applications, notably in the administration of medicinal products (in nanospheres or nanocapsules, via the oral route or the transdermal route, etc.). The hydrogels according to the present invention may also be used to fight skin burns. They may also be used for a wide range of applications in the clinical trials of experimental medicine comprising: tissue engineering and regenerative medicine, diagnostics, cell immobilization, biomolecule or cell separation, the use of “barrier” materials to regulate biological adhesion.

Thus, the object of the present invention concerns a process for producing hydrogels as defined herein characterized in that group A of formula (I) of the hydrogel as defined is selected from proteins, peptides such as peptide sequences derived from collagens, for example sequences comprising Pro-Hyp-Gly or Pro-Pro-Gly or Asp-Pro-Gly or Pro-Lys-Gly tripeptide repeats, self-assembly peptide sequences such as Arg-Ala-Asp-Ala (Seq ID 4), oligoprolines, oligoalanines, polysaccharides, such as hyaluronic acid* and derivatives thereof, oligonucleotides, C₁-C₆-alkylene-glycol polymers, or polyvinylpyrrolidone. * Hyaluronic acid may be used for the structuring properties thereof and/or for the biological activities thereof.

The object of the present invention further relates to a process for producing a hydrogel as defined above, characterized in that group B of formula (II) of the hydrogel as defined herein is a biomolecule selected from a peptide, an oligopeptide, a protein, such as collagen, a deoxyribonucleic acid, a ribonucleic acid, a polysaccharide, such as a pectin, a chitosan, a hyaluronic acid, a polyarabinose and polygalactose polysaccharide and a glycolipid.

Preferably, fragment B of formula (II) is an antimicrobial, antibiotic and/or antifungal agent listed above. More preferably, fragment B of formula (II) is more particularly an antimicrobial peptide selected from amphipathics, cationics, daptomycin, polymyxin, tachyplesin, magainin, defensins, cathelicidins, histatins, cecropins, melittin, temporins, bombinins.

The object of the present invention further relates to a process for producing a hydrogel as defined above, characterized in that the sol-gel polymerization process is carried out at physiological pH, i.e., at a pH ranging between pH 6 and 9, preferentially between pH 7 and 8, and even more preferentially at pH 7.4±0.1, or in that the hydrogel is formed in the presence of a sufficient amount of water so that the water content of the hydrogel is at least 50 wt. % relative to the total weight of the hydrogel formed.

The object of the present invention further relates to a process for producing a hydrogel as defined above, characterized in that the hydrogel is formed in the presence of a sufficient amount of water so that the water content of the hydrogel is at least 50 wt. % relative to the total weight of the hydrogel formed. Advantageously, the hydrogel according to the present invention may contain between 60% and 99% water by weight relative to the total weight of the hydrogel formed according to the present invention, more advantageously the hydrogel according to the present invention may contain between 70% and 98% water by weight and even more advantageously the hydrogel according to the present invention may contain between 75% and 97%, indeed between 80% and 95% water by weight relative to the total weight of the hydrogel formed.

The object of the present invention also relates to a process for producing a hydrogel as defined above, characterized in that said hydrogel is polymerized on or in at least a first hydrogel as a support, thus resulting in a multi-layer hydrogel. Any technique known to the person skilled in the art for obtaining a multi-layer is applicable in the present case. Simply, a first hydrogel may be cast into a mold bottom, then a second hydrogel cast onto this first hydrogel and so on. Furthermore, the hydrogels according to the present invention may be cut and shaped as desired; this cutting may take place before the casting of an additional hydrogel onto the first cut/shaped hydrogel or multi-layer.

The object of the present invention further relates to a hydrogel for therapeutic and/or surgical use, as described above, characterized in that it allows the delivery and/or the transport of active molecules, or for use in vivo in tissue engineering.

### FIGURES

FIG. 1: this figure relates to cytotoxicity tests on L929 fibroblasts. The “TC-PS” control (“low control” column in the figure) represents cell viability on TC-PS (i.e., without hydrogel). The “lysed cells” control (“high control” column in the figure) corresponds to complete lysis of the cells and thus to maximum toxicity. The “PLA-50” control makes it possible to confirm cell viability in the presence of poly(lactic acid). The “2.5% NaF silylated PEG hydrogel” sample concerns cells incubated 24 h in the presence of a hydrogel containing 10% silylated PEG by mass obtained with 2.5% NaF by weight. The “0.3% NaF silylated PEG hydrogel” sample concerns cells incubated 24 h in the presence of a hydrogel containing 10% silylated PEG by mass obtained with 0.3% NaF by weight.

FIG. 2: this figure concerns tests to quantify the release of fluorescein from various hydrogels according to the invention. The hydrogels grafted with fluorescein by covalent bond show much lower release rates than the hydrogel containing fluorescein simply enclosed with no covalent bond.

FIG. 3: this figure concerns tests of cell adhesion on the hydrogels according to the present invention. Two controls were selected for comparison: one indicates the measured fluorescence values of the culture medium in the absence of cells and the other is the fluorescence measured for the cells deposited directly on TC-PS. On this comparative basis, three series of tests were carried out with silylated PEG hydrogels according to the invention with 10% bare silylated PEG by mass, then with 7.5% molar or 15% molar (relative to the number of moles of silylated PEG) of a cell adhesion peptide (“RGD”) linked by covalent bond to said hydrogel.

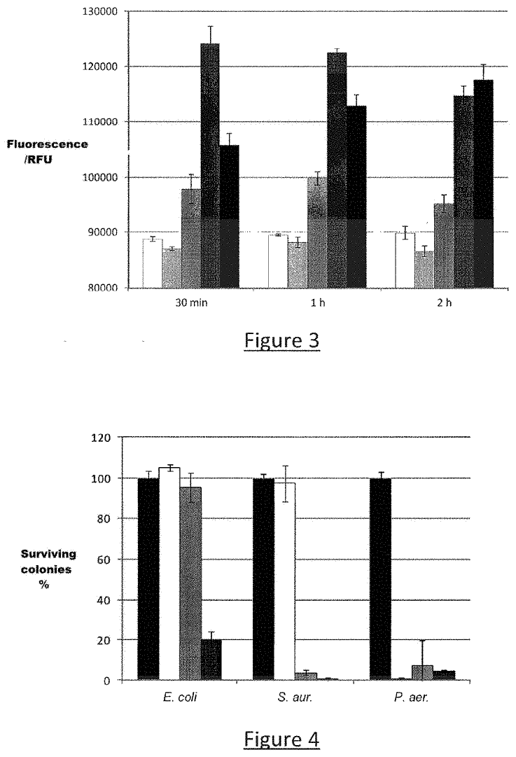

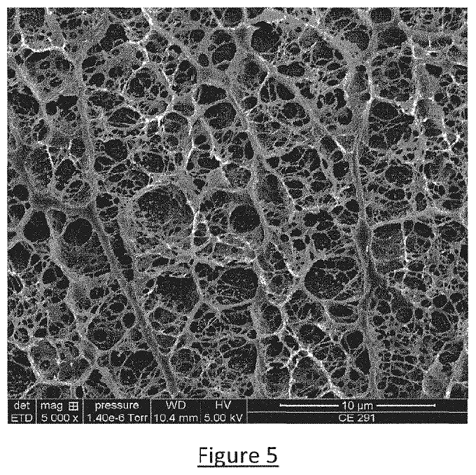

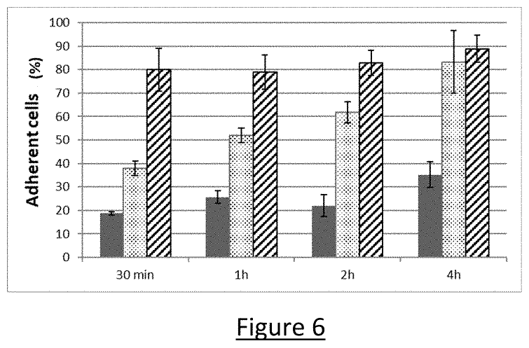

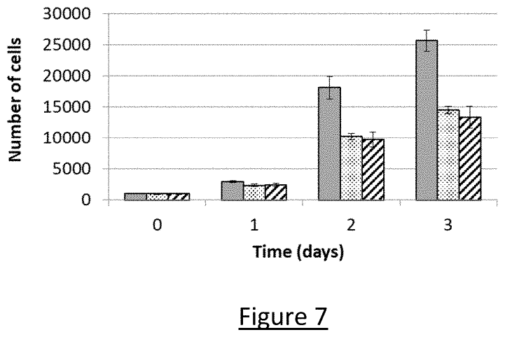

Caption for FIG. 3:
Columns (of the histogram) in white (i.e., the first columns starting from the left of the histogram): culture medium in the absence of cells;
Columns in light gray (i.e., the second columns starting from the left of the histogram): cells deposited on bare PEG hydrogel;
Columns in medium gray (i.e., the third columns starting from the left of the histogram): cells deposited on PEG hydrogel containing 7.5% RGD hybrid peptide;
Columns in dark gray (i.e., the fourth columns starting from the left of the histogram): cells deposited on PEG hydrogel containing 15% RGD hybrid peptide;
Columns in black (i.e., the fifth columns starting from the left of the histogram): cells deposited on TCPS.

FIG. 4: this figure concerns antibacterial tests with hydrogels according to the present invention. The bacteria which were the subject of the test are E. coli, S. aureus and P. aeruginosa. Two control tests were carried out: one being an evaluation of the number of bacterial colonies on simple agar gel, the other on bare silylated PEG hydrogel according to the present invention (i.e., a silylated PEG hydrogel with 10% silylated PEG by mass). An antibacterial peptide was grafted onto this same gel (i.e., bare PEG hydrogel) by covalent bonds in various proportions (7.5 mol. % of antibacterial peptide and 15 mol. % of antibacterial peptide relative to the number of moles of silylated PEG). The bacterial colonies which were formed in contact with the gets are counted after 24 h of incubation at 37° C.

Caption for FIG. 4:
Columns (of the histogram) in black (i.e., the first columns starting from the left of the histogram): colonies seeded on agar;
Columns in white (i.e., the second columns starting from the left of the histogram): colonies seeded on bare PEG hydrogel;
Columns in light gray (i.e., the third columns starting from the left of the histogram): colonies seeded on PEG hydrogel with 7.5% antibacterial hybrid peptide;
Columns in dark gray (i.e., the fourth columns starting from the left of the histogram): colonies seeded on PEG hydrogel with 15% antibacterial hybrid peptide.

FIG. 5: illustrates a Cryo-SEM view of the hydrogel containing the bi-silylated peptide prepared.

FIG. 6: illustrates the adhesion of murine mesenchymal stem cells (mMSC) on the hydrogel according to the invention, on collagen foams and on TC-PS. The dark gray histograms represent the TC-PS support. The histograms with black dots represent the hydrogel support according to the invention. The histograms with black diagonal lines represent the collagen-type commercial support.

FIG. 7: illustrates the proliferation of mMSC on the hydrogel according to the invention, on the collagen foam and on TC-PS. The caption for the histograms is the same as for FIG. 6.

### EXAMPLES

The examples below in no way limit the scope of the protection sought and are provided by way of illustration according to the present invention.

### Abbreviations

ACN, acetonitrile; Ahx, ε-aminohexanoic acid; Boc, t-Butyloxycarbonyl; DCM, dichloromethane; DIEA, diisopropylethylamine; DMF, N—N′-dimethylformamide; DPBS, Dulbecco's phosphate buffered saline; ESI-MS, electrospray ionization mass spectrometry; Fmoc, fluorenylmethoxycarbonyl; HBTU, —N,N,N′,N′-tetramethyl-O-(1H-benzotriazol-1-yl)uronium hexafluorophosphate; HPLC, high-performance liquid chromatography; HRMS, high-resolution mass spectrometry; LC/MS, mass spectrometry coupled with liquid chromatography; Pbf, 2,2,4,6,7-pentamethyldihydrobenzofuran-5-sulfonyl; PEG, polyethylene glycol; pip, piperidine; PS, polystyrene; NMR, nuclear magnetic resonance; RT, room temperature, i.e., ranging between 20 and 25° C.; TFA, trifluoroacetic acid; THF, tetrahydrofuran; TIS, triisopropylsilane.

Materials and Methods

Analytical HPLCs were carried out on an Agilent Infinity 1260 apparatus equipped with a diode array and a Kinetex C₁₈ reversed-phase column, 2.6 μm, 50×4.6 mm with a gradient of 0% to 90% (% by volume) of B in 5 min with eluent A: water/0.1% TFA and eluent B: ACN/0.1% TFA with a flow rate of 2.5 mL/min.

Purifications by preparative HPLC were carried out on a Waters HPLC 4000 apparatus, equipped with a UV 486 detector and a Waters DeltaPak C₁₈ reversed-phase column, 40×100 mm, 100 Å, 15 μm, with a flow rate of 50 mL/min. The solvents used are H₂O/0.1% TFA and ACN/0.1% TFA.

Samples for LC/MS analyses were prepared in a water/ACN mixture (50:50, v/v) containing 0.1% TFA. The LC/MS device consists of a Waters Alliance 2695 HPLC, coupled to a Water Micromass ZQ spectrometer (electrospray ionization, positive mode). Analyses were carried out with a Phenomenex Onyx reversed-phase column, 25×4.6 mm with a flow rate of 3 mL/min with a gradient of 0% to 100% (vol. %) of B in 2.5 min with eluent A: water/0.1% HCO₂H; eluent B: ACN/0.1% HCO₂H. UV detection was set at 214 nm. Mass spectra were acquired with a solvent flow rate of 200 μL/min. Nitrogen is used as the nebulizing and drying gas. Data are obtained by scanning m/z from 100 to 1000 in 0.75 seconds or from 200 to 1600 in 0.9 seconds. High-resolution mass spectrometry analyses were carried out in positive mode on a time-of-flight (TOF) spectrometer equipped with an electrospray ionization source.

¹H, ¹³C and ²⁹Si NMR spectra were recorded at room temperature (RT) in deuterated solvents on a spectrometer at 400, 101 and 79 MHz, respectively. Chemical shifts (δ) are given in parts per million using the residual non-deuterated solvents as references (CHCl₃ in CDCl₃, δH=7.26 ppm; DMSO-d6, δH=2.50 ppm). Signals are designated s (singlet), d (doublet), t (triplet), q (quadruplet), dt (doublet of triplets), m (multiplet), etc. Coupling constants are measured in hertz.

Attachment of an Amino Acid to the 2-Chlorotrityl Chloride Resin:

The 2-chlorotrityl chloride resin (1.44 mmol Cl/g, 1 eq) is placed in a solid-phase peptide synthesis reactor equipped with a sintered glass. The protected amino acid Fmoc-AA-OH (3 eq) is coupled to the resin in the presence of DIEA (5 eq) in DMF overnight. After standard washings (3×DMF, 1×MeOH and 1×DCM), the Fmoc-AA-Cltrityl resin is dried under vacuum for 12 h. Resin load is determined by 299 nm detection of the piperidine-dibenzofulvene adduct which is formed in the pip/DMF (20:80 v/v) deprotection solution.

Fmoc Deprotection:

The Fmoc-Rink Amide AM PS resin or Fmoc-peptidyl-resin is placed in a solid-phase reactor equipped with a sintered glass. The Fmoc group is removed by two successive treatments with a DMF-piperidine solution (80:20; v/v, 2×20 min). Between the two treatments, the solution is filtered and replaced with fresh solution. The conventional washing steps are carried out at the conclusion of the deprotection (3×DMF, 1×MeOH and 1×DCM).

Coupling of an Acid Amino:

The amino acid N-ter protected with an Fmoc group (3 eq) is dissolved in DMF (10 mL per g of resin) in the presence of HBTU (3 eq) and DIEA (3 eq) for 10 min. This solution is added to the peptidyl-resin the N-ter of which is free. The resin is stirred at RT for 1 h 30 min then washed (3×DMF, 1×MeOH and 1×DCM).

### Example 1: Silylation of PEG

Polyethylene glycol with an average molecular mass of 2000 g/mol (2.00 g, 1.00 mmol) is dried under vacuum at 80° C. overnight then dissolved in anhydrous THF (12 mL) under argon. Triethylamine (1.66 mL, 12 mmol, 12 eq) and isocyanatopropyl-triethoxysilane (744 μL, 3 mmol, 3 eq) are added. The mixture is refluxed for 48 h then concentrated under reduced pressure. The bi-silylated PEG is then precipitated in hexane. After centrifugation, it is washed 3 times with hexane and dried under vacuum. It is obtained in the form of a white powder stored at 4° C. under argon. ¹H NMR (400 MHz, CDCl₃) δ 5.00 (sl, 2H, NH), 4.18 (t, J=4.7 Hz, 4H, H-6), 3.79 (q, J=7.0 Hz, 12H, H-2), 3.62 (s, 177H, CH₂ PEG), 3.14 (dd, J=13.2, 6.7 Hz, 4H, H-5), 1.58 (qu, J=7.7 Hz, 4H, H-4), 1.20 (t, J=7.0 Hz, 18H, H-1), 0.64-0.54 (m, 4H, H-3). ¹³C NMR (101 MHz, CDCl₃) δ 156.72 (C), 70.55 (CH₂), 63.76 (CH₂), 58.26 (CH₂), 43.29 (CH₂), 23.12 (CH₂), 18.38 (CH₃), 9.12 (CH₂). ²⁹Si NMR (79 MHz, CDCl₃) δ −45.73 (s).

### Example 2: Synthesis of a Peptide Derived from Collagen and Bi-Silylated

Preparation of the Tripeptide: Fmoc-Pro-Hyp-Gly-OBzl

Coupling of Boc-Hyp-OH:

Into a 5000-mL single-neck round-bottom flask are introduced H-Gly-OBzl.HCl (11.42 g, 56.6 mmol, 1 eq) dissolved in AcN, and DIEA (37.44 mL, 226.5 mmol, 4 eq). In a beaker, Boc-Hyp-OH (13.3 g, 56.6 mmol) is dissolved in AcN. DIEA and pyBOP (29.3 g, 56.63 mmol, 1 eq) are added to this solution. The two amino acids are contacted and stirred at RT for 4 h. The reaction is monitored by analytical HPLC.

Once the reaction ends, AcN is evaporated and the orange oil obtained is solubilized in ethyl acetate. This solution is washed with aqueous solutions of KHSO₄, NaHCO₃ and NaCl. The organic phase is then dried with MgSO₄ and the solvent evaporated under reduced pressure.

N-Ter Deprotection of the Dipeptide:

The product is then dissolved in 150 mL of TFA for 40 min until gas evolution has completely ceased. The TFA is then evaporated under reduced pressure and the dipeptide is precipitated in diethyl ether then freeze-dried.

Coupling of Fmoc-Pro-OH:

Fmoc-Pro-OH is coupled to H-Hyp-Gly-OBzl according to the same protocol as the coupling of Boc-Hyp-OH described above. The reaction lasts 2 h. At the conclusion of the washings, the tripeptide is purified on silica gel (Biotage apparatus, SNAP 340 g column, gradient of 0% MeOH in DCM to 10% MeOH in DCM, product eluted at 50% of the gradient, 71% yield).

C-Ter Deprotection:

Into a 250-mL single-neck round-bottom flask are introduced Fmoc-Pro-Hyp-Gly-OBzl (20.4 g, 40.2 mmol) dissolved in EtOH and 200 mg of Pd/C. The whole is placed under hydrogen bubbling for 6 h at 60° C. The reaction is monitored by analytical HPLC. Once the reaction ends, the solution is filtered through Celite then concentrated under reduced pressure. The solid is taken up in H₂O/AcN 50:50 v/v and freeze-dried (Yield: 86%).

Synthesis of Peptide Ac-Lys-(Pro-Hyp-Gly)₃-Lys-NH₂ (Seq ID 5) on a Support Starting with the Tripeptide Block

Synthesis is carried out in a syringe equipped with a sintered glass on a Rink Amide PS resin the load of which is 0.94 mmol/g with a 1-mmol synthesis scale. The resin is swollen in DCM then washed with DMF. It is deprotected by means of two treatments with pip/DMF deprotection solution (15 mL for 5 min, 3 washings with DMF, 15 mL for 20 min then washings (3×DMF, 3×DCM, 1×DMF)). The usual coupling conditions are slightly modified. The couplings are extended to 2 h and are carried out with 1.5 eq of Fmoc-Lys(Boc)-OH or Fmoc-(Pro-Hyp-Gly)-OH, 5 eq of DIEA and 1.5 eq of HATU which replaces HBTU. At the end of the coupling, the resin is washed with the following solvents: (3×DMF, 3×DCM, 1×DMF). The last coupling is also followed by deprotection of the Fmoc group. Next, the peptidyl-resin is acetylated with acetic acid (2 eq) in DCM (10 mL) in the presence of BOP (2 eq) and DIEA (4 eq) for 1 h 30 min. The resin is washed then cleaved in a TFA/TIS/H₂O mixture (95:2.5:2.5 v/v/v, 50 ml). The “cleavage” solution is concentrated under reduced pressure and the peptide is precipitated with ether. After centrifugation and removal of the supernatant, the crude peptide is taken up in a water/ACN mixture and freeze-dried. Finally, it is purified by preparative HPLC on a Luna C₁₈ reversed-phase column (15 μm, 250×50 mm) with a flow rate of 120 mL/min with a gradient of 0% to 6% of B in 6 min, of 6% to 10% of B in 8 min and of 10% to 18% in 24 min with eluent A: H₂O/0.1% TFA and eluent B: ACN/0.1% TFA. Yield: 49%; purity >99%. LC/MS (ESI⁺): t_{R}=0.73 min, 1118 ([M+H]⁺, 5%), 559 ([M+2H]²⁺, 100).

Silylation

Ac-Lys-(Pro-Hyp-Gly)₃-Lys-NH₂ (20 mg, 14.9 μmol) is dissolved in anhydrous dimethylformamide (300 μL) under argon. Diisopropylethylamine (12.4 μL, 71.3 μmol, 4.8 eq) and then 3-isocyanatopropyltriethoxysilane (9.7 μL, 39.3 μmol, 2.6 eq) are added to the nonapeptide solution. The reaction mixture is left 50 min under stirring. The end of the reaction is monitored by LC/MS. The solvent is evaporated under reduced pressure. Next, the silylated nonapeptide is precipitated with diethyl ether. After centrifugation, the hybrid peptide is washed 3 times with diethyl ether then the powder obtained is dried under vacuum. LC/MS (ESI⁺): t_{R}=0.82 min, 704 ([M+2H-2H₂O]²⁺, 100%), 695 ([M+2H-3H₂O]]²⁺, 80) and t_{R}=0.87 min (conformer), 704 ([M+2H-2H₂0]²⁺, 70%), 695 ([M+2H-3H₂0]]²⁺, 100).

### Example 3: Preparation of a Hydrogel Composed of a Molecule of Formula (I)

A molecule of formula (I), for example the bi-silylated PEG prepared in example 1 or the collagen-derived bi-silylated peptide synthesized in example 2, is dissolved in pH 7.4 phosphate buffer (DPBS Dulbecco's phosphate buffered saline), preferably at a concentration of 10% by mass, in the presence of sodium fluoride (3 mg of NaF per mL of DPBS). The non-viscous solution is incubated at 37° C.; a get then forms. The gelation time depends on the nature and the concentration of the molecule of formula (I).

### Example 4: Synthesis of Silylated Peptide (EtO)₃Si—(CH₂)₃—NHCO-(βAla)₄-Gly-Ara-Gly-Asp-Ser-Pro-OH (Seq ID 6)

H-(βAla)₄-Gly-Arg-Gly-Asp-Ser-Pro-OH (Seq ID 6) is synthesized on a 2-chlorotrityl chloride resin (load: 1.44 mmol/g, synthesis scale: 0.5 mmol) using an Fmoc/tBu strategy. The amino acids used are successively Fmoc-Pro-OH, Fmoc-Ser(tBu)-OH, Fmoc-Asp-(tBu)-OH, Fmoc-Gly-OH, Fmoc-Arg(Pbf)-OH, Fmoc-Gly-OH and Fmoc-βAla-OH four times. Each amino acid coupling is followed by N-ter deprotection of the Fmoc group. “Cleavage” of the peptide and deprotection of the side chains are carried out in a TFA/TIS/H₂O mixture, 95:2.5:2.5 v/v/v, for 4 h. After precipitation, the peptide is purified by preparative HPLC on a C₁₈ column. It is obtained after freeze-drying with 63% yield. It is then silylated by reaction with 3-isocyanatopropyltriethoxysilane (1.1 eq) in DMF at a concentration of 30 mM in the presence of DIEA (3 eq). The reaction is monitored by analytical HPLC. The DMF is then evaporated under reduced pressure and the silylated peptide is precipitated in diethyl ether. It is recovered by centrifugation at the conclusion of 3 washings in diethyl ether.

¹H NMR (400 MHz, DMSO-d6) δ 8.61-8.40 (m, 2H, NH Asp and Gly), 8.21-8.04 (m, 2H, NH Gly N-ter and Arg), 7.99-7.78 (m, 3H, NH βAla)), 7.58-7.42 (m, 1H, NH Ser), 7.34-6.98 (m, 3H, OH Ser, COOH Asp and C-ter), 5.97 (t, J=5.6 Hz, 1H, NH urea), 5.78 (t, J=5.6 Hz, 1H, NH urea βAla), 4.56 (q, J=6.9 Hz, 1H, Hα Ser), 4.49-4.38 (m, 1H, Hα Asp), 4.38-4.26 (m, 1H, Hα Arg), 4.22 (dd, J=8.7, 4.1 Hz, 1H, Hα Pro), 4.02-3.75 (m, 4H, Hα Gly), 3.73 (q, J=6.9 Hz, 6H, CH₂ ethoxy), 3.67-3.46 (m, 4H, Hδ Pro and Hβ Ser), 3.46-3.28 (m, 2H, Hδ Arg), 3.28-3.11 (m, 8H, Hβ BAla), 2.92 (q, J=6.1 Hz, 2H, H-3), 2.60-2.48 (m, 2H, Hβ Asp), 2.29 (t, J=7.1 Hz, 2H, Hα βAla), 2.26-2.09 (m, 6H, Hα βAla), 1.87 (dd, J=13.3, 6.5 Hz, 2H, Hγ Pro), 1.60-1.56 (m, 2H, Hβ Arg), 1.55-1.42 (m, 2H, Hδ Arg), 1.38 (m, 2H, H-2), 1.19-1.09 (t, J=7.2 Hz, 9H, CH₃ ethoxy), 0.55-0.42 (m, 2H, H-1). ¹³C NMR (101 MHz, DMSO-d6) δ 174.27 (C), 172.82 (C), 171.38 (C), 171.27 (C), 171.14 (C), 170.86 (C), 170.80 (C), 169.58 (C), 169.43 (C), 169.02 (C), 168.77 (C), 158.41 (C), 157.52 (C), 62.14 (CH₂), 59.54 (CH), 58.14 (CH₂), 53.51 (CH), 52.60 (CH), 49.78 (CH), 47.02 (CH₂), 42.46 (CH₂), 42.36 (CH₂), 40.89 (CH₂), 36.68 (CH₂), 36.30 (CH₂), 35.88 (CH₂), 35.76 (CH₂), 30.10 (CH₂), 29.13 (CH₂), 25.39 (CH₂), 24.91 (CH₂), 24.01 (CH₂), 18.68 (CH₃), 7.72 (CH₂). ²⁹Si NMR (79 MHz, DMSO-d6) δ −45.10. LC/MS (ESI⁺): Only the hydrolysis products of the ethoxysilane groups to silanols are detected. t_{R}=0.62 min, 1035 ([M+H]⁺, 50%), 509 ([M+H—OH]²⁺, 60), 500 ([M-2OH]²⁺, 100). HRMS: 1119.5481. C₄₅H₇₄N₁₈O₁₄Si implies [M+H]⁺, 1119.5479.

### Example 5: Synthesis of Silylated Peptide HO(CH₃)₂Si—(CH₂)₃—NHCO-Ahx-Arg-Ara-NH₂

Antibacterial peptide H-Ahx-Arg-Arg-NH₂ is synthesized on a Rink Amide resin (load 0.94 mmol/g, synthesis scale: 3 mmol) using an Fmoc/tBu strategy. The amino acids used are successively Fmoc-Arg(Pbf)-OH twice and Fmoc-ε-aminohexanoic acid. Each coupling is followed by N-ter deprotection of the Fmoc group. The tripeptide is then silylated on a support in DMF (10 mL/g resin) by using 3-isocyanatopropyldimethylchlorosilane (3 eq) in the presence of DIEA (3 eq). The silylation reaction is left under stirring overnight then the resin is washed (3×DMF, 1×MeOH and 1×DCM) and cleaved in TFA for 5 h. The “cleavage” solution is concentrated under reduced pressure then the silylated antibacterial peptide is precipitated in diethyl ether and finally purified by preparative HPLC on a C₁₈ column (Eluent A: H₂O/0.1% TFA, eluent B: ACN/0.1% TFA, gradient: 0% to 7% of B in 2 min then 7% to 30% of B of 23 min, the product eluted at 16% of B). After freeze-drying, the silylated antibacterial peptide is obtained in the form of a white powder (Yield: 68%, purity >98%). ¹H NMR (400 MHz, D₂O) δ 4.19 (ddd, J=14.4, 8.6, 5.7 Hz, 2H, Hα Arg), 3.09 (t, J=6.9 Hz, 4H, Hδ Arg), 2.96 (td, J=6.8, 1.9 Hz, 4H, H-3 and H-4), 2.17 (t, J=7.3 Hz, 2H, H-8), 1.82-1.59 (m, 4H, Hβ Arg), 1.59-1.44 (m, 6H, H-7 and Hγ Arg), 1.41-1.31 (m, 4H, H-2 and H-5), 1.24-1.12 (m, 2H, H-6), 0.53-0.41 (m, 2H, H-1), 0.00 (s, 6H, Si(CH₃)₂). ¹³C NMR (101 MHz, DMSO-d6) δ 172.40 (C), 171.76 (C), 170.69 (C), 157.38 (C), 156.00 (C), 51.38 (CH), 51.02 (CH), 41.54 (CH₂), 34.28 (CH₂), 29.01 (CH₂), 28.34 (CH₂), 28.08 (CH₂), 25.24 (CH₂), 24.24 (CH₂), 23.16 (CH₂), 14.24 (CH₂), −0.51 (CH₃). ²⁹Si NMR (79 MHz, DMSO-d6) δ 7.97 (dimer). LC/MS (ESI⁺): t_{R}=0.71 min, 602 ([M+H]⁺, 10%), 302 ([M+2H]²⁺, 100), 293 ([M+2H—NH₃]²⁺, 30). HRMS: 602.3926. C₂₄H₅₁N₁₁O₅Si implies [M+H]⁺, 602.3922.

### Example 6: Synthesis of a Hydroxydimethylsilyl Fluorescein

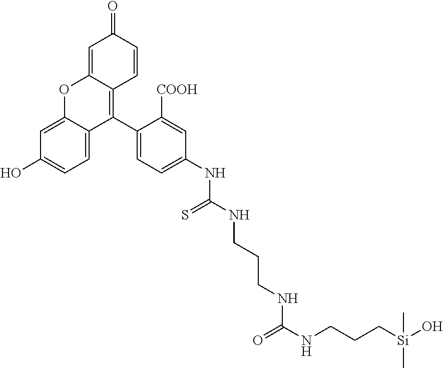 [SMILES: C[Si](C)(O)CCCNC(=O)NCCCNC(=S)Nc1ccc(-c2c3ccc(=O)cc-3oc3cc(O)ccc23)c(C(=O)O)c1]

N-Boc-1,3-propanediamine (56.4 mg, 0.324 mmol, 1.05 eq) is added to a solution of fluorescein isothiocyanate (FITC, 120 mg, 0.308 mmol) in anhydrous DMF (3 mL) in the presence of DIEA (100 μL). The reaction mixture is stirred for 1 h at RT under argon. Next, the DMF is evaporated under reduced pressure. The Boc-amino fluorescein is precipitated and washed in diethyl ether then dried. It is then solubilized in TFA (4 mL) and this solution is stirred for 1 h. The reaction mixture is concentrated and precipitated in diethyl ether. After centrifugation, the supernatant is removed. The fluorescein amine is purified by preparative HPLC on a C₁₈ column (Eluent A: H₂O/0.1% TFA, eluent B: ACN/0.1% TFA, gradient: 0% to 15% of eluent B in 3 min then 15% to 40% of eluent B in 25 min, the product eluted at 22% of eluent B) and obtained in the form of TFA salt (180 mg, 100%, purity=95%).

3-Isocyanatopropylchlorodimethylsilane (16.2 μL, 0.0910 mmol, 1.05 eq) is added to a solution of fluorescein amine purified beforehand (50.0 mg, 0.0867 mmol) in anhydrous DMF (2 mL) in the presence of DIEA (45.2 μL, 0.260 mmol, 3 eq). The reaction mixture is stirred under argon for 1 h. The solvent is evaporated under reduced pressure and the crude product is obtained by precipitation in diethyl ether. The hydroxydimethylsilyl fluorescein is purified by preparative HPLC on a C₁₈ column (Eluent A: H₂O/0.1% TFA, eluent B: ACN/0.1% TFA, gradient: 0% to 20% of eluent B in 4 min, 20% to 26% of eluent B in 6 min then 26% to 46% of eluent B in 30 min, the product eluted at 32% of eluent B, yield: 42%, purity: 97%) ¹H NMR (400 MHz, DMSO-d6) δ 10.01 (sl, 1H, COOH), 9.97 (s, 1H, NH thiourea from FITC), 8.20 (s, 1H, H-4), 8.12 (s, 1H, NH thiourea), 7.71 (d, J=7.6 Hz, 1H, H-6), 7.14 (d, J=8.3 Hz, 1H, H-5), 6.65 (d, J=2.2 Hz, 2H, H-9 and H-10), 6.59-6.51 (m, 4H, H-7, H-8, H-11 and H-12), 5.88 (s br, 2H, NH urea), 3.43-3.38 (m, 2H, H-3), 3.03 (t, J=6.5 Hz, 2H, H-1), 2.92 (t, J=6.9 Hz, 2H, H-3′), 1.62 (qu, J=6.5 Hz, 2H, H-2), 1.37-1.29 (m, 2H, H-2′), 0.47-0.36 (m, 2H, H-1′), 0.00 and −0.04 (2 s, 6H, H-4′ of the dimer and of the monomer, respectively). ¹³C NMR (101 MHz, DMSO-d6) δ 179.51 (C), 167.65 (C), 158.62 (C), 157.51 (C), 151.02 (C), 140.47 (C), 128.75 (CH), 128.19 (CH), 125.68 (C), 123.18 (CH), 116.66 (C), 115.79 (CH), 114.24 (C), 111.71 (CH), 108.87 (C), 101.36 (CH), 41.55 (CH₂), 40.52 (CH₂), 35.85 (CH₂), 28.80 (CH₂), 23.13 (CH₂), 14.20 (CH₂), −0.51 (CH₃), −0.75 (CH₃). ²⁹Si NMR (79 MHz, DMSO-d6) δ 11.24 (monomer), 7.99 (dimer). LC/MS (ESI⁺): t_{R}=1.33 min, 623 ([M+H]⁺, 60%), 390 ([M+H—NH₂(CH₂)₃NH—CONH(CH₂)₃Si(CH₃)₂OH]⁺, 15), 312 ([M+2H]²⁺, 60), 303 ([M+2H—H₂O]²⁺, 100. HRMS: 623.1996. C₃₀H₃₄N₄O₇SSi implies [M+H]⁺, 623.19%.

### Example 7: Preparation of Hydrogels Comprising a Molecule of Formula (I) and a Molecule of Formula (II)

A molecule of formula (I), for example the bi-silylated PEG prepared in example 1 or the collagen-derived bi-silylated peptide synthesized in example 2, is dissolved in pH 7.4 phosphate buffer (DPBS Dulbecco's phosphate buffered saline), preferably at a concentration of 10% by mass, in the presence of sodium fluoride (3 mg of NaF per mL of DPBS). A molecule of formula (II), for example the silylated peptide containing sequence Arg-Gly-Asp synthesized in example 4 or the antibacterial silylated peptide prepared in example 5 or the silylated fluorescein described in example 6, is added to the solution of the molecule of formula (I) at a concentration ranging between 1% and 15 mol. % relative to the molecule of formula (I). The non-viscous solution is incubated at 37° C.; a get then forms. The gelation time depends on the nature and the concentration of the selected molecules.

### Example 8: Examples of Applications of a Hydrogel According to the Invention

Optimization of Synthesis

The bifunctional unit (EtO)₃—Si—(CH₂)₃—NHCO-(PEG2000)-OCONH—(CH₂)₃—Si—(OEt)₃ (i.e., “bi-silylated hybrid PEG block”) was synthesized by reacting polyethylene glycol (MW=2000 Da) with 3-isocyanatopropyltriethoxysilane. Next, the bi-silylated hybrid PEG block was engaged in the sol-get process, consisting of hydrolysis of ethoxysilyls to silanols and the condensation thereof to form siloxane bonds. This process was carried out at 37° C., at pH 7.2-7.4 in phosphate buffer (DPBS). Sodium fluoride (NaF) was used as nucleophilic catalyst to accelerate the condensation reactions. Various concentrations of bi-silylated hybrid PEG and of sodium fluoride were tested, thus showing their influence on gelation time (table 1):

TABLE 1
Gelation time of solutions of bi-silylated hybrid PEG in DPBS at
37° C. and viscoelastic moduli of the hydrogels obtained

| Composition of the gel | | | | |
|---|---|---|---|---|
| Bi-silylated | | | | |
| hybrid | | Gelation | | |
| PEG block | NaF | time | G′ | G″ |
| (% by mass) | (% by mass) | (min) | (Pa) | (Pa) |
| 20 | 5.0 | 10 | n.d. | n.d. |
| 20 | 2.5 | 15 | 77750 | 204 |
| 10 | 5.0 | 20 | n.d. | n.d. |
| 10 | 2.5 | 35 | 18960 | 49 |
| 10 | 0.3 | 120 | 9947 | 61 |
| 5 | 5.0 | 50 | n.d. | n.d. |
| 5 | 2.5 | 220 | 5413 | 39 |

The effect of the PEG/water ratio and of the NaF concentration on the mechanical properties of the hydrogels was also studied. The viscoelastic response of the hydrogels was measured in oscillation mode using an AR 2000 rheometer (TA Instruments, Inc.) with a parallel geometry of 20 mm diameter (normal force=2 N). Changes in the storage (G′) and loss (G″) moduli were measured as a function of oscillation frequency within the linear viscoelastic range (0.1% deformation, from 0.01 Hz to 10 Hz) for gels of different compositions. The moduli values for a 10 Hz frequency are presented in table 1. All of the samples exhibit the properties of a solid with G′ greater than G″. The storage moduli were used as measurement of the elasticity of the hydrogels. They remained stable for one week. Extending from 5000 to 80000 Pa depending on the composition of the gel, they encompass a wide range of stiffness. Very few variations were observed on the loss moduli for all samples. Thus, according to the application concerned, the stiffness of the gets can be adjusted by varying the bi-silylated hybrid PEG and/or NaF concentrations.

Cytotoxicity Tests of the Bare Hydrogel

Cytotoxicity tests were carried out on two of the hydrogels presented above each containing 10% bi-silylated hybrid PEG by mass relative to the mass of solvent and 0.3% or 2.5% NaF by mass, respectively.

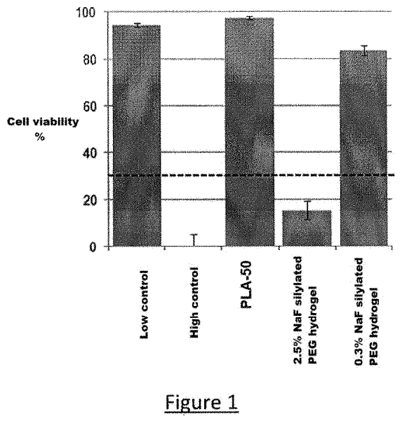

Line L929 murine fibroblasts were seeded in tissue culture-treated polystyrene wells. After 24 h of proliferation, these cells were incubated with the hydrogels for a further 24 h. Cytotoxicity was then measured using a test showing the release of lactate dehydrogenase by the cells. As expected, the hydrogels containing the highest NaF concentration proved toxic. However, more than 80% cell viability was observed for an NaF concentration of 3 mg/mL, which means that the latter hydrogel is not toxic to the cells (FIG. 1).

These results were confirmed by microscopic observations which showed healthy spindle-shaped cells.

Verification of the Functionalization of the Hydrogel

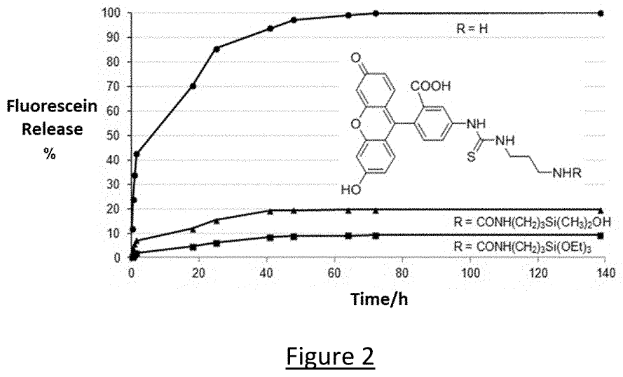

In order to show the covalent incorporation of a (bio)molecule into the gel and the absence of release over time of the grafted molecules, fluorescein derivatives were selected to be chemically linked to the hydrogels in accordance with the present invention (see FIG. 2 for the exact formulae). Thus, if there were to be a release of the grafted molecules, the use of fluorescein would make it possible to easily detect it.

Fluorescein isothiocyanate (FITC) was used to prepare two types of fluorescein derivatives giving covalent bonds (triethoxysilane and dimethylhydroxysilane), as well as a non-silylated molecule used as control. Each fluorescein derivative was dissolved at a concentration of 5.2 mM in a solution of bi-silylated hybrid PEG, itself at a concentration of 10% by mass relative to the mass of DPBS used as solvent. Sodium fluoride was added, and the solutions were homogenized. Fluorescent hybrid hydrogels were obtained at 37° C. in 30 minutes. The various hydrogels were placed in phosphate buffer (10 mL) and fluorescein release was monitored by HPLC. In the case of non-covalent enclosure, complete release of fluorescein was observed within 72 h. As expected, fluorescein release is limited (relative to the control) in the case of the covalent derivatives, and reaches a plateau after 72 hours. This indicates the stability of the hybrid covalent bonds. A maximum release of 9% and 20% (relative to the total amount of fluorescein introduced) was observed for the hydrogels obtained with “triethoxysilylfluorescein” and “dimethythydroxyfluorescein”, respectively (FIG. 2). This release may be attributed to the hybrid molecules trapped non-covalently, which could not have reacted during the gelation process. This is consistent with the fact that the triethoxysilyl derivative should be more reactive than dimethylhydroxysilyl during the condensation reaction.

Cell Adhesion Test

A peptide containing the RGD sequence was selected to promote cell adherence. It is sequence GRGDSP (SEQ ID 7).

The deprotected peptide H-GRGDSP-OH (Seq ID 7) was first prepared by peptide synthesis on a solid support using an Fmoc/tBu strategy and functionalized with a triethoxysilyl group using 3-isocyanatopropyttriethoxysilane (ICPTES). A solution of bi-silylated hybrid PEG at 10% by mass relative to the mass of DPBS containing 0.3% NaF by mass was prepared and the silylated GRGDSP hybrid peptide was added to this solution. The relative concentration of silylated GRGDSP in the mixture before reaction was set at 7.5% (first solution) and 15% (second solution) in moles relative to the number of moles of bi-silylated hybrid PEG. These two solutions were placed at 37° C. overnight, to provide two “silylated-PEG/RGD” hydrogels.

L929 fibroblasts were seeded on the surface of the silylated-PEG/RGD hydrogels and on a non-functionalized hybrid PEG hydrogel (“bare hydrogel”). Adherent cells after 30 min, 1 h and 2 h of incubation were detected and assayed using the PrestoBlue Cell Viability Reagent® (FIG. 3). No cell adhesion was observed on the bare hydrogel. On the other hand, cell adhesion was very effective in the case of the hydrogel containing 15% molar silylated RGD (solution 2). Indeed, adhesion on the latter after 30 min of incubation is better than on the tissue culture-treated polystyrene (TC-PS), and this for adhesion surfaces of identical size.

Antibacterial Tests

Likewise, hydrogels with antibacterial properties were prepared by using the peptide sequence H-Ahx-Arg-Arg-NH₂ suitably silylated on the N-terminal side. To that end, the peptide H-Ahx-Arg(Pbf)-Arg(Pbf)-NH— on Rink amide resin was functionalized at the N-terminal end with a dimethylhydroxysilyl group before cleavage of the resin and deprotection of the side chains. The resulting hybrid peptide was added to solutions of bi-silylated hybrid PEG according to the protocol described above for the “silylated PEG-RGD” hydrogel. The antibacterial activity of the hydrogels was evaluated against Escherichia coli, Staphylococcus aureus and Pseudomonas aeruginosa. The hydrogels were surface-inoculated with bacteria and covered with trypticase soy agar. After 24 h of incubation at 37° C., the bacterial colonies were counted (FIG. 4). The “bare” PEG hydrogels seem to inhibit the growth of P. aeruginosa even in the absence of peptide. For E. coli and S. aureus, the antibacterial effect was provided by the grafted peptide. Indeed, 15 mol. % antibacterial peptide induced complete inhibition of S. aureus growth and reduced E. coli growth by 80%.

Adhesion and Cell Proliferation Test

A hydrogel as obtained in Example 3 above, containing a bi-silylated hybrid peptide illustrated below, was prepared:

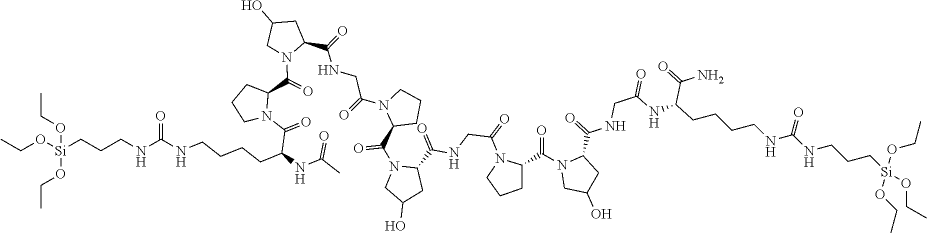 [SMILES: CCO[Si](CCCNC(=O)NCCCC[C@H](NC(=O)CNC(=O)[C@@H]1CC(O)CN1C(=O)[C@@H]1CCCN1C(=O)CNC(=O)[C@@H]1CC(O)CN1C(=O)[C@@H]1CCCN1C(=O)CNC(=O)[C@@H]1CC(O)CN1C(=O)[C@@H]1CCCN1C(=O)[C@H](CCCCNC(=O)NCCC[Si](OCC)(OCC)OCC)NC(C)=O)C(N)=O)(OCC)OCC]

It was able to be shown that this hydrogel has an alveolar internal structure which may be favorable to cell proliferation. Cryo-scanning electron microscopy (SEM) analyses show a multimicrometric alveolar system.

This is indeed quite visible in FIG. 5, which shows a Cryo-SEM view of the hydrogel containing the bi-silylated peptide prepared.

Cell Adhesion

It was also shown that the hydrogel obtained allows adhesion of murine mesenchymal stem cells (mMSC) more effectively than the tissue culture-treated polystyrene and after 4 h as effectively as a commercial collagen foam.

50 μL of a cell suspension at 60,000 cells per mL was deposited on various materials, including the hydrogel according to the invention as described in this section. Cell culture-treated polystyrene (TC-PS) and a commercial foam of purified and cross-linked bovine type I collagen were used as controls.

Various adhesion times at 37° C. were studied before removing the medium, rinsing with DPBS then counting the cells by means of a CellTiter-Glo viability test. The results are reported in FIG. 6.

FIG. 6 shows the adhesion of mMSC on the hydrogel according to the invention, on collagen foams and on TC-PS.

Cell Proliferation

The hydrogel is as good a support as the collagen foams for cell proliferation.

1000 mMSC were deposited on various materials, including the hydrogel according to the invention. Cell culture-treated polystyrene (TC-PS) and a commercial foam of purified and cross-linked bovine type I collagen were used as controls.

Each day for 3 days, the culture medium of a sample series is replaced, and the cells are counted by means of a CellTiter-Glo viability test. The results are reported in FIG. 7.

FIG. 7 indeed shows the proliferation of murine mesenchymal stem cells (mMSC) on the hydrogel according to the invention, on collagen foam and on TC-PS.

Cells Survival

The hydrogel allows satisfactory survival of enclosed cells for at least 25 hours.

A major advantage of the process described in the present patent is the possibility of adding cells to the still-liquid mixture of silylated precursors. Thus, the gel forms without addition of additional chemical reagent, while enclosing the cells. Murine mesenchymal stem cells were thus encapsulated for 25 hours with excellent viability, comparable to the positive control of cells cultured in 2D on a TC-PS surface.

The encapsulation protocol is as follows. A solution of hybrid hydrogel is prepared by solubilization of 30 mg of the hybrid peptide of example 3, the structure of which is indicated above, in 250 μL of DMEM culture medium containing 4.5 g/L glucose and 0.12 mg/mL NaF. The solution is incubated at 37° C. for 17 h 15 min. At that time, the solution is still liquid, but its viscosity has increased. 50 μL of a suspension of mMSC at 500,000 cells per mL in DMEM medium is added. The concentration of bi-silylated hybrid peptide is then 10% by mass. The hybrid solution is homogenized and 30 μL of this solution is deposited in the wells of a 96-well cell culture plate. The gel gradually forms at 37° C. 25 hours later, a solution of Calcein-AM and Ethidium homodimer III in DPBS is added to the gels and the latter are analyzed by confocal microscopy.

It is noted that most of the cells enclosed within the hydrogel according to the invention were stained with calcein and are thus alive. The viability of the cells enclosed within the gel is comparable to the viability of the cells deposited on TC-PS.

### CONCLUSION

The ease of synthesis and of use of the hydrogels according to the present invention was shown. These hydrogels were usefully employed with molecules of diverse chemical structures, proving the versatility of the process according to the present invention. Hydrogels exhibiting satisfactory rheological, biological and/or physicochemical properties were thus able to be prepared with very little variation (indeed no variation) of the operating conditions, apart from the nature of the grafted molecules.

1.-7. (canceled)

8. A process for producing a hydrogel comprising the steps of:
a) sol-gel polymerization of at least one molecule of formula (I): 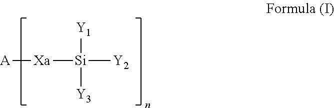
wherein:
n is an integer greater than or equal to 2;
A is a structural organic polymer, preferentially of synthetic origin which may be, for example, selected from proteins, peptides such as collagen derivatives, in particular the sequences comprising Pro-Hyp-Gly or Pro-Pro-Gly or Asp-Pro-Gly or Pro-Lys-Gly tripeptide repeats, self-assembly peptide sequences such as Arg-Ala-Asp-Ala (SEQ ID 4), oligoprolines, oligoalanines, polysaccharides, such as hyaluronic acid and derivatives thereof, oligonucleotides, C₁-C₆-alkylene-glycol polymers, or polyvinylpyrrolidone;
Xa is a chemical bond or a spacer group preferentially represented by a divalent radical derived from a saturated or unsaturated aliphatic hydrocarbon chain comprising from 1 to 10 carbon atoms, optionally intercalated with one or more structural linkers selected from arylene or fragments —O—, —S—, —C(═O)—, SO₂ or —N(R₁)—, wherein said chain is unsubstituted or is substituted by one or more radicals selected from halogen atoms, a hydroxyl group, a C₁-C₄ alkyl group, a benzyl group and/or a phenethyl group;
R₁ represents a hydrogen atom, an aliphatic hydrocarbon group comprising from 1 to 6 carbon atoms, a benzyl or a phenethyl;
Y₁, Y₂, Y₃, which may be identical or different, each independently represents a hydrogen atom, a halogen atom, an —OR₂ group, an aryl or a saturated or unsaturated aliphatic hydrocarbon chain comprising from 1 to 6 carbon atoms optionally substituted by a halogen atom, an aryl group or a hydroxyl group;
R₂ represents a hydrogen atom, an aryl group or a saturated or unsaturated aliphatic hydrocarbon chain comprising from 1 to 6 carbon atoms;
wherein at least two Xa groups as defined above are linked to different attachment points on A;
b) mixing with water, optionally at the same time as step a); and
c) recovering the hydrogel.

9. The process for producing a hydrogel according to claim 8, wherein said process comprises the addition, at the same time as or subsequent to step a), of at least one type of molecule of formula (II): 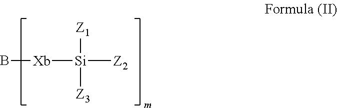
wherein:
m is an integer greater than or equal to 1, preferentially equal to 1;
B is an active ingredient, preferentially a biomolecule or a fluorophore, which may be, for example, selected from a peptide, an oligopeptide, a protein, such as collagen, a deoxyribonucleic acid, a ribonucleic acid, a polysaccharide, such as a pectin, a chitosan, a hyaluronic acid, a polyarabinose and polygalactose polysaccharide, and a glycolipid;
Xb is a chemical bond or a spacer group preferentially represented by a divalent radical derived from a saturated or unsaturated aliphatic hydrocarbon chain comprising from 1 to 10 carbon atoms, optionally intercalated with one or more structural linkers selected from arylene or fragments —O—, —S—, —C(═O)—, SO₂ or —N(R₃)—, wherein said chain is unsubstituted or is substituted by one or more radicals selected from halogen atoms, a hydroxyl group, a C₁-C₄ alkyl group, a benzyl group and/or a phenethyl group;
R₃ represents a hydrogen atom, an aliphatic hydrocarbon group comprising from 1 to 6 carbon atoms, a benzyl or a phenethyl;
Z₁, Z₂, Z₃, which may be identical or different, each independently represents a hydrogen atom, a halogen atom, an —OR₄ group, an aryl or a saturated or unsaturated aliphatic hydrocarbon chain comprising from 1 to 6 carbon atoms optionally substituted by a halogen atom, an aryl group or a hydroxyl group;
R₄ represents a hydrogen atom, an aryl group or a saturated or unsaturated aliphatic hydrocarbon chain comprising from 1 to 6 carbon atoms; and
wherein preferentially only one of the Z₁, Z₂, or Z₃ groups is a halogen atom or an OR₄ group.

10. The process for producing a hydrogel according to claim 8, wherein the sol-gel polymerization process is carried out at physiological pH or in that the hydrogel is formed in the presence of a sufficient amount of water so that the water content of the hydrogel is at least 50 wt. % relative to the total weight of the hydrogel formed.

11. The process for producing a hydrogel according to claim 8, wherein said hydrogel is polymerized on or in at least a first hydrogel as a support, thus resulting in a multi-layer hydrogel.

12. A hydrogel that can be obtained by the process according to claim 8.

13. Therapeutic and/or surgical method comprising the use of hydrogel according to claim 12 in a patient in need thereof.

14. In vitro tissue engineering method comprising the use of a hydrogel according to claim 12.

15. The process for producing a hydrogel according to claim 9, wherein the sol-gel polymerization process is carried out at physiological pH or in that the hydrogel is formed in the presence of a sufficient amount of water so that the water content of the hydrogel is at least 50 wt. % relative to the total weight of the hydrogel formed.

16. The process for producing a hydrogel according to claim 9, wherein said hydrogel is polymerized on or in at least a first hydrogel as a support, thus resulting in a multi-layer hydrogel.

17. The process according to claim 8, wherein the molecule of formula (I) is prepared in homogeneous conditions.

18. The process according to claim 9, wherein the molecule of formula (II) is prepared in homogeneous conditions.

19. The process according to claim 8, wherein the polymerization is carried out in situ in a living organism.

20. The process according to claim 9, wherein the polymerization is carried out in situ in a living organism.
